(19) United States
(12) Patent Application Publication
(10) Pub. No.: US 2007/0190536 A1
(43) Pub. Date: Aug. 16, 2007

(54) Method for using biomaterials as reagent for nano-patterning

(75) Inventors: Chii-Dong Chen, Taipei Hsien (TW); Hung-Yi Lin, Tainan (TW); Pei-Yin Chi, Ping-Dong City (TW); Li-Chu Tsai, Taipei Hsien (TW)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Academia Sinica

(21) Appl. No.: 11/355,146

(22) Filed: Feb. 16, 2006

(51) Int. Cl. C12Q 1/68; C12M 3/00; B05D 3/00

(52) U.S. Cl. 435/6; 435/287.2; 427/2.11; 977/924

(57) ABSTRACT

A pattern transfer method includes providing a substrate, forming a first biomaterial over the substrate, exposing the first biomaterial to a pattern writing agent in a manner consistent with a pattern to be transferred, forming a second biomaterial over the first biomaterial, wherein the second biomaterial reacts and bonds with portions of the first biomaterial not exposed to the pattern writing agent, and does not react and bond with portions of the first biomaterial exposed to the pattern writing agent.

### FIELD OF THE INVENTION

This invention relates in general to a method for nano-patterning using biomaterials and, more particularly, to a lithography method of using biomaterials instead of conventional resist as a reagent.

### BACKGROUND OF THE INVENTION

Lithography is a process used in semiconductor device fabrication to transfer a pattern to the surface of a substrate such as semiconductor wafer or glass. In photolithography, the pattern is held in a photomask and transferred to the substrate using a photoresist as a reagent. Electron beam (e-beam) lithography is a maskless process, in which the pattern is directly written onto the substrate by controlling a machine to generate a beam of electrons and bombard the surface of the substrate with the e-beam in a manner consistent with the pattern. The process of e-beam lithography is illustrated in FIGS. 1A-1C.

In FIG. 1A, a substrate 100 is provided. An e-beam resist 102 such as polymethyl-methacrylate (PMMA) is spun onto substrate 100. In FIG. 1B, substrate 100 with resist 102 formed thereon is exposed with an e-beam 104. E-beam 104 generated by an e-beam machine (not shown), such as a field-emission scanning electron microscope based e-beam writer, scans the surface of substrate 100 in a predetermined manner and bombards resist 102 in certain areas but not other areas. The pattern in which e-beam 104 scans the surface of substrate 100 may be controlled by software operating the e-beam machine. Then, in FIG. 1C, resist 102 is developed in a suitable developer solution. Those areas bombarded by e-beam 104 are dissolved in the developer solution, resulting in openings 106 in resist 102. As a result, resist 102 is patterned. The pattern in resist 102 may be transferred to another layer of material. For example, the pattern may be transferred to substrate 100 by etching substrate 100 using resist 102 as a mask. The pattern may also be transferred to a layer of material such as metal subsequently formed on resist 102 through a lift-off process.

A problem with conventional lithography processes is that conventional resists such as PMMA used in e-beam lithography are toxic to biological materials and the conventional lithography processes are therefore undesirable for processing biological devices. Moreover, curing and chemical etching may be required for using conventional resists, and these processes increase the uncertainty of end products.

### SUMMARY OF THE INVENTION

A pattern transfer method consistent with embodiments of the present invention includes providing a substrate, forming a first biomaterial over the substrate, exposing the first biomaterial to a pattern writing agent in a manner consistent with a pattern to be transferred, and forming a second biomaterial over the first biomaterial, wherein the second biomaterial reacts and bonds with portions of the first biomaterial not exposed to the pattern writing agent, and does not react and bond with portions of the first biomaterial exposed to the pattern writing agent.

Additional features and advantages of the invention will be set forth in part in the description which follows, and in part will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are incorporated in and constitute a part of this specification, illustrate embodiments of the invention and, together with the description, serve to explain the features, advantages, and principles of the invention.

In the drawings,

FIGS. 1A-1C illustrate a conventional e-beam process;

FIGS. 2A-2E illustrate an e-beam patterning method consistent with embodiments of the present invention;

FIGS. 3A-3B show images of patterns realized by methods consistent with embodiments of the present invention; and

FIG. 4 shows a relationship between intensities of the images of FIGS. 3A-3B and doses of an e-beam used in generating the patterns of FIGS. 3A-3B.

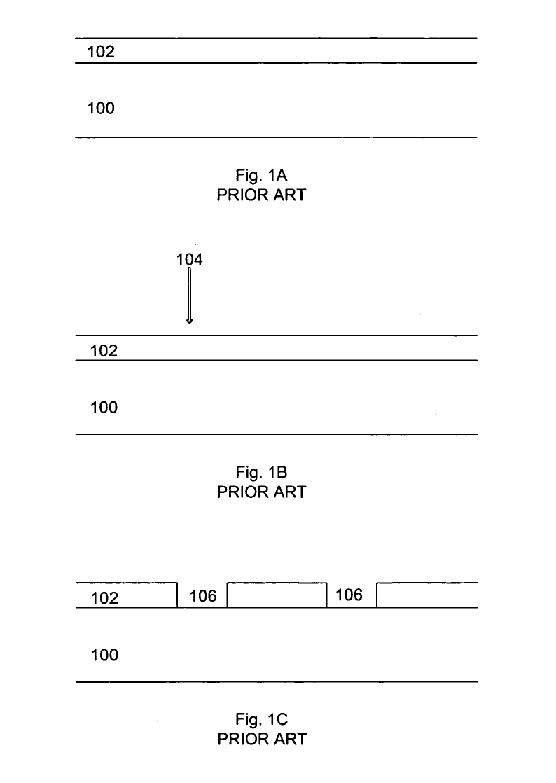

### DESCRIPTION OF THE EMBODIMENTS

Reference will now be made in detail to the present embodiments of the invention, examples of which are illustrated in the accompanying drawings. Wherever possible, the same reference numbers will be used throughout the drawings to refer to the same or like parts.

Consistent with embodiments of the present invention, there are provided methods of using biomaterials instead of conventional resist for transferring patterns. Particularly, a first biomaterial and a second biomaterial that can bond with each other through reaction are used. The first biomaterial comprises a material that changes properties under exposure to a pattern writing agent such that, after the exposure, the first biomaterial does not react with the second biomaterial. The pattern writing agent may be a UV beam, X-ray, a beam of irradiation particles, or a beam of electrons (e-beam). First, the first biomaterial is formed on a substrate and exposed to the pattern writing agent in a manner consistent with a pattern. Then, the second biomaterial is deposited on the first biomaterial and allowed to react with the first biomaterial. Because the second biomaterial only bonds with portions of the first biomaterial not exposed to the pattern writing agent, the pattern is transferred to the second biomaterial.

By labeling the second biomaterial with a certain material, the pattern may be further transferred onto that certain material. For example, if a fluorescent material is used to label the second biomaterial, a fluorescent pattern is formed. Alternatively, if the second material is labeled with gold, a gold pattern is formed.

A specific example is given below, where DNA (deoxyribonucleic acid) molecules are used as the first and second biomaterials.

DNA has been proposed as a template for assembling nanostructures to produce optical, electrical, or other types of functional circuits. For example, metal has been grown on a DNA backbone to form nanowires and quantum dots. DNA has also been used as a template for field-effect transistors.

A DNA molecule is a polymer which can be single stranded or double stranded. In a double-stranded DNA, two complementary strands of DNA bond to each other via hydrogen bonds between corresponding nucleotides on the two strands. Typically, a DNA nucleotide may have one of four different bases: adenine (A), guanine (G), cytosine (C), and thymine (T), respectively referred to as nucleotide A, nucleotide G, nucleotide C, nucleotide T. Nucleotide A bonds with nucleotide T, and nucleotide G bonds with nucleotide C. Thus, a chain of nucleotides, generally referred to as an oligonucleotide, may hybridize with another oligonucleotide if corresponding nucleotides complement each other, and such oligonucleotides are said to be complementary of each other. For example, an oligonucleotide including nucleotides A may hybridize with an oligonucleotide including nucleotides T, and an oligonucleotide including nucleotides C may hybridize with an oligonucleotide including nucleotides G. Additionally, an oligonucleotide may include more than one type of nucleotide.

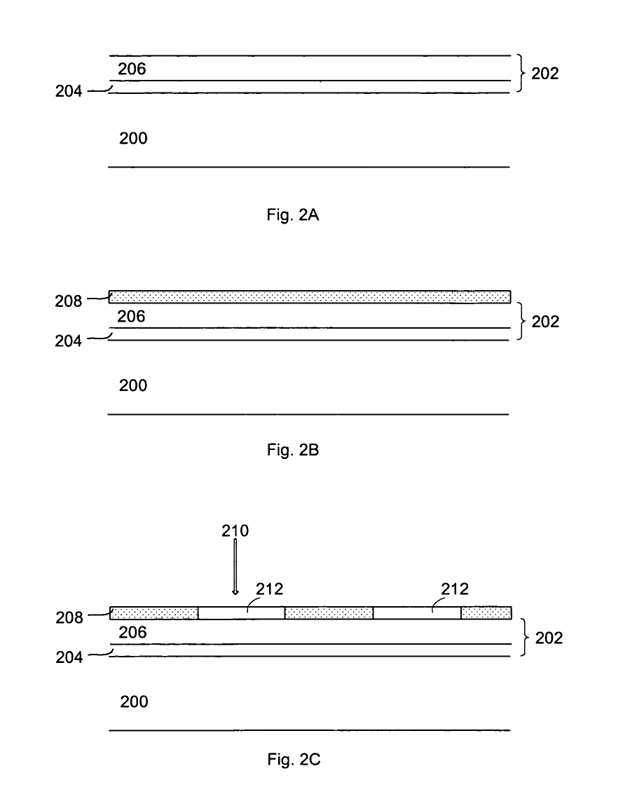

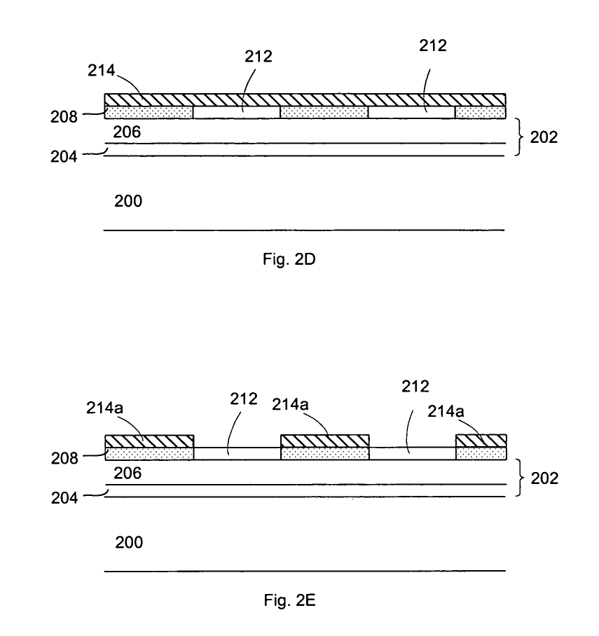

After being bombarded by e-beam, some oligonucleotides show inhibition to hybridizing with their complementary oligonucleotides. For example, oligonucleotides comprising nucleotides T, referred to as poly(T) oligonucleotides, after exposure to e-beam, show a certain degree of inhibition to hybridizing with oligonucleotides comprising nucleotides A, referred to as poly(A) oligonucleotides. Thus, consistent with embodiments of the present invention, the first biomaterial may comprise poly(T) oligonucleotides, the second biomaterial may comprise poly(A) oligonucleotides, and the pattern writing agent may be e-beam. FIGS. 2A-2E illustrate a method using poly(T) and poly(A) oligonucleotides for transferring a pattern.

In FIG. 2A, a substrate 200 is cleaned with acetone or isopropyl acetone and an immobilizing film 202 is deposited on substrate 200. Substrate 200 may comprise any suitable material, such as semiconductor, glass, or sapphire. Immobilizing film 202 may comprise any material that provides a mechanism for immobilizing oligonucleotides to be deposited thereon. For example, immobilizing film 202 may comprise a thin chromium (Cr) film 204 and a thin gold (Au) film 206 sequentially deposited on substrate 200 through thermal evaporation, where Au film 206 is cleaned by oxygen plasma in a reactive ion etcher for 2 minutes to improve a hydrophilic characteristic of Au film 206.

In FIG. 2B, modified poly(T) oligonucleotides 208 are deposited on immobilizing film 202 and become immobilized. Immobilization may be carried out at room temperature for a period of time. Modified poly(T) oligonucleotides 208 comprise oligonucleotides modified in such a manner as to bond with immobilizing film 202 and become immobilized. For convenience of illustration, the term “oligonucleotide” is used to refer to both modified and non-modified oligonucleotides throughout this specification. For example, when immobilizing film 202 comprises Cr film 204 and Au film 206, modified poly(T) oligonucleotides 208 may comprise thiolated T-based ssDNA (single-stranded DNA), such that sulphur in modified poly(T) oligonucleotides 208 may bond with the gold in Au film 206 to immobilize modified poly(T) oligonucleotides 208. After immobilization, substrate 200 having immobilizing film 202 and modified poly(T) oligonucleotides 208 deposited thereon is rinsed with DI water and blow-dried with nitrogen gas.

In FIG. 2C, an e-beam 210 generated by an e-beam machine (not shown), such as a field-emission scanning electron microscope based e-beam writer, exposes modified poly(T) oligonucleotides 208. Software such as a CAD (computer-aided design) program may operate the e-beam machine and control e-beam 210 to scan the surface of modified poly(T) oligonucleotides 208 and to write a pattern thereon. In FIG. 2C, portions 212 of modified poly(T) oligonucleotides 208 are shown to have been exposed with e-beam 210.

In FIG. 2D, probe oligonucleotides 214 comprising poly(A) are provided to hybridize with modified poly(T) oligonucleotides 208. For example, a solution containing probe oligonucleotides 214 may be dropped onto substrate 200. Modified poly(T) oligonucleotides 208 in portions 212, which were bombarded by e-beam 210, show inhibited hybridization with probe oligonucleotides 214, while the remaining portions of modified poly(T) oligonucleotides 208 hybridize with probe oligonucleotides 214.

Finally, as FIG. 2E shows, non-bonded probe oligonucleotides 214 are removed by washing in, e.g., DI water. Thus, the pattern written onto modified poly(T) oligonucleotides 208 is transferred onto probe oligonucleotides 214a that remain.

By labeling probe oligonucleotides 214a with a certain material, the pattern in modified poly(T) oligonucleotides 208 may be indirectly transferred onto that certain material. For example, if probe oligonucleotides 214a are labeled with a fluorescent material, patterned fluorescence appears. Alternatively, if probe oligonucleotides 214a are labeled with gold, patterned gold may be formed. Other materials, such as other noble metals, semiconductor colloidal nanoparticles, e.g., CdSe, CdS, etc., may be used to label probe oligonucleotides 214a and to form desired nanostructures. Labeling may be performed either before or after the removal of the non-bonded probe oligonucleotides 214.

Experiments have been performed to form both a fluorescent pattern and a gold pattern using the above method. Particularly, a sample was prepared on a glass. The glass was first cleaned with acetone and isopropanol, rinsed in DI (deionized) water, and dried in an oven. Thermal evaporation was performed to deposit a thin Cr film and a thin Au film on the glass. The Cr film had a thickness of 50 nm and the Au film had a thickness of 350 nm. Then, the surface of the gold film was cleaned by oxygen plasma in a reactive ion etcher to improve a hydrophilic characteristic thereof. Thiolated ssDNA (5′-HS-(CH₂)₆-(T)₂₀-3′), denoted as HS-20T, having a concentration of 10 μM in a 1.0 M KH₂PO₄ solution, which may be purchased from MDBio, Inc., was deposited on the gold film and allowed to immobilize for a half day as the sulphur in HS-20T bonded with the gold in the gold film. After immobilization, the sample was rinsed in DI water and blow-dried with nitrogen gas. Then, e-beam was performed to write a pattern onto the HS-20T. The e-beam was produced by a converted field-emission scanning electron microscope (FEI Sirion 200) operated at 30 KeV and a beam current of approximately 20 pA.

To form a fluorescent pattern, 5′ Hex-dye labeled poly(A) oligonucleotides (5′-Hex-(A)₂₀-3′) having a concentration of 10 μM were provided to hybridize with the HS-20T. The hybridization was carried out in a TE-1 M NaCl solution (10 mM Tris-HCl, 1 mM EDTA, and 1 M NaCl) at room temperature for one day. The sample was then rinsed in DI water to remove non-bonded poly(A) oligonucleotides.

To form a gold pattern, biotin-modified poly(A) oligonucleotides (10 μM biotin-20A; MDBio, Inc.) were provided to hybridize with the HS-20T. After hybridization, the sample was treated with 0.1 mg/ml streptavidin (Sigma-Aldrich Co.), washed with DI water, and then treated with concentrated Au particles for 10 minutes. The sample was then washed again with DI water and blow-dried. The diameter of the Au particles was about 13 nm. Due to the high affinity of streptavidin to both biotin and Au particles, a layer of gold particles was formed where biotin-20A had bonded with the HS-20T.

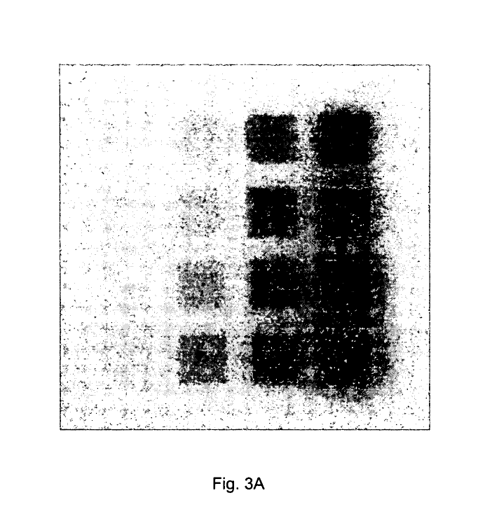

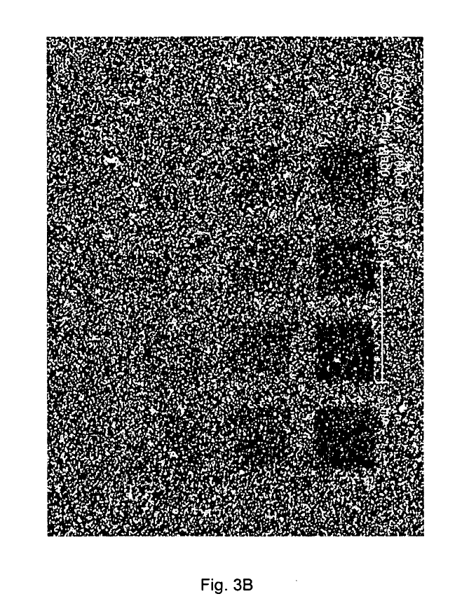

Images of the fluorescent pattern were obtained using an inverted fluorescence microscope such as Olympus IX71 equipped with a high-resolution CCD camera such as a Sony D70 camera. Images of the gold pattern were obtained by scanning electron microscopy (SEM). FIG. 3A shows an image of the fluorescent pattern and FIG. 3B shows an SEM image of the gold pattern.

In both FIGS. 3A and 3B, dark squares correspond to portions of the HS-20T exposed to the e-beam and an intensity gradient of the squares was found to correlate with doses of the e-beam. Particularly, where the dose of the e-beam was lower, hybridization was more complete and the intensity was higher. Where the dose of the e-beam was higher, hybridization was less complete and the intensity was lower.

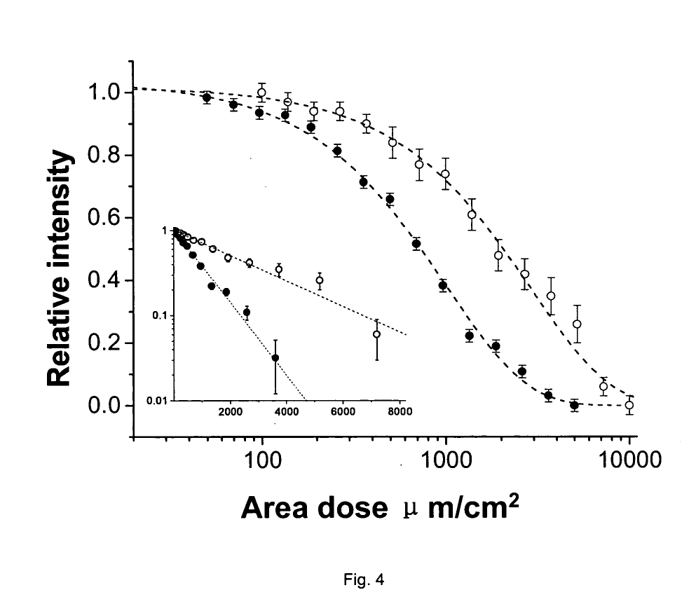

FIG. 4 shows the intensities on the images of FIGS. 3A and 3B as functions of the dose of the e-beam. Solid dots represent the fluorescent image of FIG. 3A, and empty dots represent the gold image of FIG. 3B. The inset in FIG. 4 plots the functions in a logarithmic scale. The abscissa represents the dose of e-beam per unit area. The ordinate represents the relative intensity, which is defined as the intensity in a square relative to the brightest and the darkest squares on the image. As FIG. 4 shows, as the dose of the e-beam increases, the intensity of the images decreases, indicating that a higher dose of e-beam results in less hybridization of the probe oligonucleotides with the poly(T) oligonucleotides.

Therefore, embodiments of the present invention also provide a method for forming a pattern having different depths, e.g., an image having non-uniform intensities, by controlling the dose of the e-beam in the above-described process, which method should now be apparent to one skilled in the art and is not described herein.

Also as FIG. 4 shows, Au particles exhibit less sensitivity to the dose of the e-beam than the fluorescent material used to dye the probe oligonucleotides. This is because Au particles have a larger mean size than the fluorescent probe, as a result of which less of the poly(T) oligonucleotides exposed to e-beam are needed for forming the same size of the Au pattern than of the fluorescent pattern.

Because poly(A) and poly(T) oligonucleotides bond with each other on a molecular level, the above-described pattern transfer method has very high resolution, e.g., nanometers. Thus, metal nanowires, quantum dots, or biological sensors may be formed by methods consistent with embodiments of the present invention. The resolution may be limited by the size of the materials used for labeling the probe oligonucleotides. For example, if the gold particles labeling the probe oligonucleotides have mean size of 13 nm in diameter, which is greater than a fluorescent material labeling the probe oligonucleotide, the gold pattern thus formed may have a lower resolution than the fluorescent pattern.

Also, because oligonucleotides are non-toxic to biological materials, the above method consistent with embodiments of the present invention is better suited for biological applications than conventional e-beam lithography.

Although only oligonucleotides containing nucleotides T and A are given above as examples of the first and second biomaterials, respectively, it is to be understood that the invention is not limited thereto. Oligonucleotides including nucleotides C and G, oligonucleotides including more than one type of nucleotides, and biomaterials other than DNA molecules may be used as well.

It will be apparent to those skilled in the art that various modifications and variations can be made in the disclosed process without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A pattern transfer method, comprising:
providing a substrate;
forming a first biomaterial over the substrate;
exposing the first biomaterial to a pattern writing agent in a manner consistent with a pattern to be transferred; and
forming a second biomaterial over the first biomaterial,
wherein the second biomaterial reacts and bonds with portions of the first biomaterial not exposed to the pattern writing agent, and does not react and bond with portions of the first biomaterial exposed to the pattern writing agent.

2. The method of claim 1, wherein providing the substrate comprises providing a semiconductor wafer, glass, or sapphire.

3. The method of claim 1, wherein providing the first biomaterial comprises providing oligonucleotides including thymine nucleotides.

4. The method of claim 1, wherein exposing the first biomaterial comprises exposing the first biomaterial to an electron beam, a UV beam, an X-ray, or a beam of irradiation particles.

5. The method of claim 1, wherein providing the second biomaterial comprises providing oligonucleotides including adenine nucleotides.

6. The method of claim 1, further comprising:
providing an immobilizing film on the substrate; and
immobilizing the first biomaterial to the immobilizing film.

7. The method of claim 6, wherein providing the first biomaterial comprises providing the first oligonucleotides as modified oligonucleotides which bond with the immobilizing film.

8. The method of claim 6, wherein providing the first biomaterial comprises providing the thiolated T-based ssDNA (single-stranded DNA).

9. The method of claim 1, further comprising labeling the second biomaterial.

10. The method of claim 9, wherein labeling the second biomaterial comprises dying the second biomaterial.

11. The method of claim 9, wherein labeling the second biomaterial comprises labeling the second biomaterial with nanoparticles, noble metals, or semiconductor colloidal nanoparticles such as CdSe or CdS.

12. The method of claim 9, wherein labeling the second biomaterial is performed before forming the second biomaterial over the first biomaterial.

13. The method of claim 9, wherein labeling the second biomaterial is performed after forming the second biomaterial over the first biomaterial.

14. A pattern transfer method, comprising:
providing first oligonucleotides over a substrate;
writing a pattern onto the first oligonucleotides; and
providing second oligonucleotides to hybridize with the first oligonucleotides.

15. The method of claim 14, wherein providing the first oligonucleotides comprises providing oligonucleotides including thymine nucleotides.

16. The method of claim 14, wherein providing the second oligonucleotides comprises providing oligonucleotides including adenine nucleotides.

17. The method of claim 14, wherein writing the pattern onto the first oligonucleotides comprises writing the pattern with an electron beam.

18. The method of claim 17, wherein the pattern has a non-uniform depth, and writing the pattern comprises writing the pattern by adjusting a dose of the electron beam.

19. The method of claim 14, further comprising:
providing an immobilizing film on the substrate; and
immobilizing the first oligonucleotides to the immobilizing film.

20. The method of claim 19, wherein providing the first oligonucleotides comprises providing the first oligonucleotides as modified oligonucleotides which bond with the immobilizing film.

21. The method of claim 19, wherein providing an immobilizing film comprises sequentially providing a thin film of chromium and a thin film of gold on the substrate.

22. The method of claim 14, further comprising labeling the second oligonucleotides.

23. The method of claim 22, wherein labeling the second oligonucleotides comprises dying the second oligonucleotides.

24. The method of claim 22, wherein labeling the second oligonucleotides comprises labeling the second oligonucleotides with nanoparticles, noble metals such as gold, or semiconductor colloidal nanoparticles such as CdSe or CdS.
